(19) United States
(12) Patent Application Publication
(10) Pub. No.: US 2006/0084903 A1
(43) Pub. Date: Apr. 20, 2006

(54) Method and apparatus for treating newborn umbilical stump

(76) Inventor: Hannah Keeley, Colonial Heights, VA (US)

Correspondence Address: HUNTON & WILLIAMS LLP;Riverfront Plaza, East Tower, 951 E. Byrd St., Richmond, VA, 23219-4074, US

(21) Appl. No.: 10/965,118

(22) Filed: Oct. 14, 2004

(51) Int. Cl. A61F 13/00

(52) U.S. Cl. 602/53

(57) ABSTRACT

A method and assembly for convenient newborn navel treatment including a disposable absorbent material saturated with a healing solution. According to embodiments of the present invention the healing solution may be selected so that it does not irritate the skin of a baby, or stain material surrounding the umbilical stump of a baby.

### BACKGROUND OF THE INVENTION

When a baby is born, the umbilical cord is clamped off and severed, leaving an attached piece of tissue, an umbilical stump, that is eventually shed. The navel area that surrounds the stump is similar to that of a wound, in which exposed tissue is in the process of healing and regenerating. Typically the umbilical stump is treated by the application of a stringent such as rubbing alcohol on the stump with a cotton ball, gauze, or other absorbent material. Another alternative common treatment is the application of iodine on the stump with an absorbent material. The goal of these treatments is to cleanse the wound, remove any residue and dry it out.

One drawback in the current treatment methods is inconvenience. The fluid and the absorbent material required for treatment of the umbilical stump are two separate products that must be used simultaneously. Therefore, they must be stored and transported together. In order to cleanse the umbilical stump both items must be present.

Another drawback to current methods of treatment of the umbilical stump is the number of steps required. Each additional step takes additional time. First, a dry absorbent piece of material, such as a cotton ball or gauze, needs to be obtained. Second, the container of fluid must be obtained and opened. Third, a portion of fluid must be dispensed onto the absorbent material. Fourth, the absorbent material saturated with the fluid is applied to the newborn navel area. Fifth, the healing solution needs to be closed and stored away. Finally, the absorbent material must be disposed of.

An additional drawback to the current treatment methods is the appropriateness of the fluid. Alcohol is frequently used for its drying and sterilizing properties. However, alcohol often burns, stings, or irritates the tender exposed skin of the baby, thereby causing discomfort. Iodine is also frequently used to treat the umbilical stump, but does not have the drying properties that are needed. Iodine also has the unfortunate drawback of staining the clothes, blankets, or other materials that may come into contact with the skin surrounding a baby's umbilical stump.

### SUMMARY OF THE INVENTION

It is an object of the present invention to provide a single assembly for the storage of both an absorbent material and a healing solution for the more convenient treatment of an umbilical stump. The present invention allows for the storage and transportation of both the absorbent material and the healing solution in a single unit, thereby minimizing the risk that one of the two necessary items will be misplaced or forgotten. This aspect of the present invention also minimizes the space required to transport the materials necessary to treat a baby's umbilical stump.

Another object of the present invention is to minimize the number of steps required and therefore the amount of time required for the treatment of an umbilical stump. While other methods require at least six steps in order to clean and treat the baby's umbilical stump, an embodiment of the present invention allows for the treatment of the umbilical stump in three simple steps. The user simply obtains an absorbent pad from the assembly, applies it to the area surrounding the baby's umbilical stump, and finally the user disposes of the absorbent pad and the assembly. An alternate embodiment of the present invention allows for the treatment of the umbilical stump in four steps. The user obtains an absorbent pad from the assembly, applies it to the area surrounding the baby's umbilical stump, and disposes of the absorbent pad, and the closes and stores the assembly.

Yet another object of the present invention is to prevent irritation of the skin surrounding a baby's umbilical stump. Healing solutions disclosed in an embodiment of the present invention are comprised of natural, mild ingredients that exhibit superior antiseptic and astringent qualities and are also mild and gentle on the skin of a newborn baby.

Still another object of the present invention is to prevent staining clothing or materials that may come into contact with the baby's skin surrounding the umbilical stump.

### BRIEF DESCRIPTION OF THE DRAWINGS

FIG. 1 is a drawing of a disposable absorbent pad.

FIG. 2 is an extrapolated view of an assembly in which disposable, absorbent pads may be packaged according to one embodiment of the present invention.

FIG. 3 is an extrapolated view of another assembly in which disposable, absorbent pads may be packaged according to one embodiment of the present invention.

FIG. 4 is a drawing of an application technique for one embodiment of the present invention.

### DETAILED DESCRIPTION OF EXEMPLARY EMBODIMENTS

Details of newborn navel healing pads according to the present invention will be more fully understood from the description given hereunder with reference to the accompanying drawings.

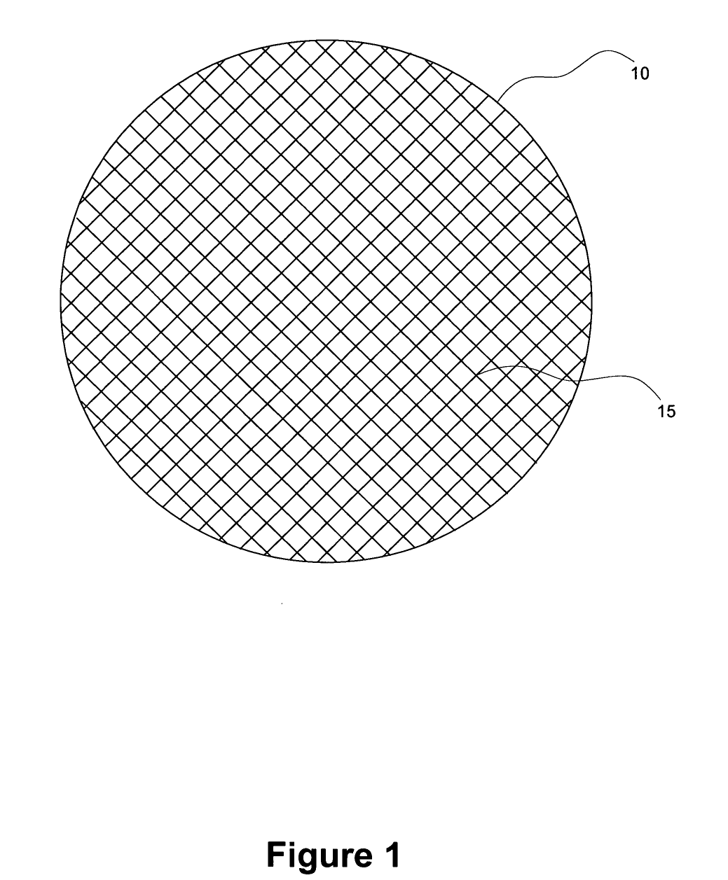

FIG. 1 is a drawing of a disposable absorbent pad. It is understood that this disposable absorbent pad 10 is only one example. The disposable absorbent pad 10 is round, nonwoven and is compressed to created a waffle-like texture 15. It is understood that for the purposes of the present invention, the disposable absorbent pad 10 may be woven or non-woven. The disposable absorbent pad 10 also may be of varying shapes or sizes. Any number of absorbent materials may be used for the disposable absorbent pad, including naturals material such as cotton or synthetic material such as rayon or polypropylene.

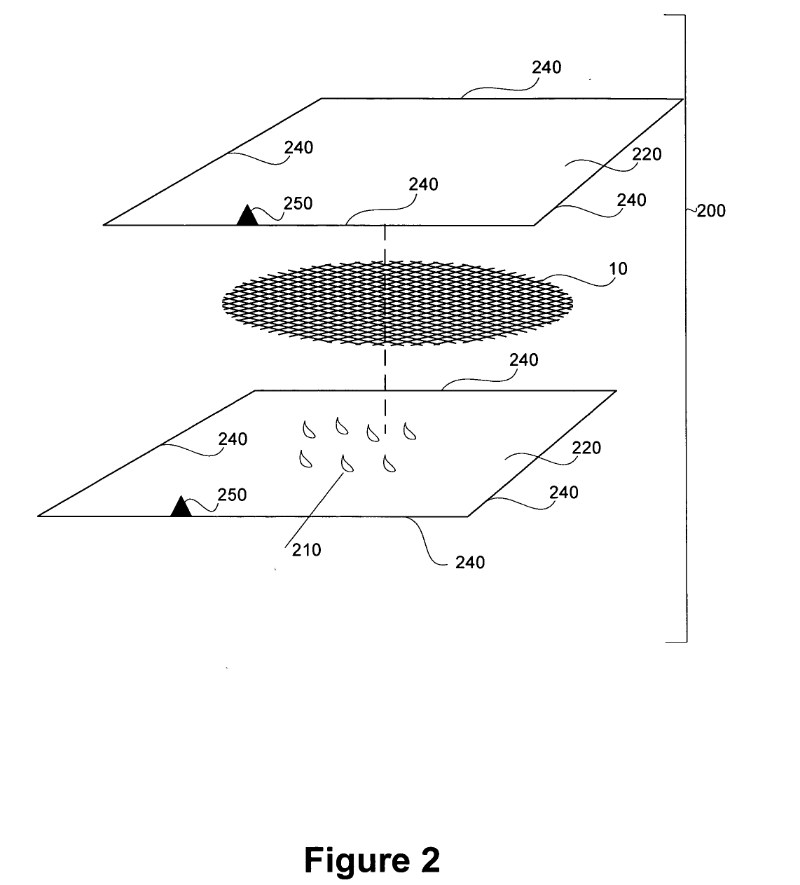

FIG. 2 is an extrapolated view of an assembly in which disposable, absorbent pads may be packaged according to one embodiment of the present invention. According to this embodiment of the present invention, the absorbent pad 10 is stored in a disposable assembly 200 with healing solution 210. The disposable assembly 200 shown in FIG. 2 may be comprised of two layers 220 sealed together around the edges in a manner that prevents the healing solution 210 from leaking out of the disposable assembly 200. The two layers 220 may be made from any material that is not permeable to the healing solution 210. In the embodiment shown in FIG. 2, the two layers 220 are squares, and all four edges 240 of the square are sealed. In one embodiment of the present invention the two layers 220 may be made of a material that is easily torn open. In another embodiment of the present invention, one or more edges 240 of the layers 220 may be indented 250, for ease of opening the assembly 200.

The healing solution 210 may be comprised of any number of ingredients. In one embodiment of the present invention the healing solution exhibits superior antiseptic and astringent qualities while also being mild and gentle on the skin of a baby. The healing solution 210 may contain a mild astringent, such as witch hazel. Other examples of mild astringents are citric acid, lemon oil, lavender oil, white grapefruit oil, rosemary oil, tea tree oil, geranium oil, and other essential oils. It is understood that any number of other fruit or herb extracts may be used as a mild astringent. Additionally, the healing solution 210 may contain a healing accelerator, application of which speeds the regeneration of skin tissue. Examples of healing accelerators are lavender, chamomile, aloe, and rose distillate. The healing solution 210 may also contain a preservative such as polysorbate 20, propylparaben, or methylparaben. Any other number of well known preservatives would also be suitable. Finally, the healing solution 210 may contain a naturally mild antibiotic, such as black willow bark extract. Any other antibiotic that is mild and does not irritate a newborn baby's skin would also be suitable.

In one embodiment of the present invention the healing solution may be 93.76288% water, 0.89868% hamamelis virginiana (witch hazel), 0.00598% anthemis nobilis (chamomile) distillate, 1.53537% rosa damascena (rose) distillate, 0.76768% lavendula angustifolia (lavender) distillate, 1.19824% aloe barbadensis (aloe) leaf juice, 0.29675% citrus limonum (lemon) essential oil, 0.29526% lavendula angustifolia (lavender) essential oil, 0.29526% citrus paradise (white grapefruit) essential oil, 0.07488% citric acid USP (fine), 0.03543% anthemis nobilis (chamomile) essential oil, 0.02511% melaleuca alternifolia (tea tree) essential oil, 0.01181% rosmarinus officinalis (rosemary) essential oil, 0.01181% pelargonium graveolens (geranium) essential oil, 0.00959% methylparaben, 0.00479% propylparaben, 0.77366% polysorbate 20, and 0.00149% salix nigra (black willow bark) extract.

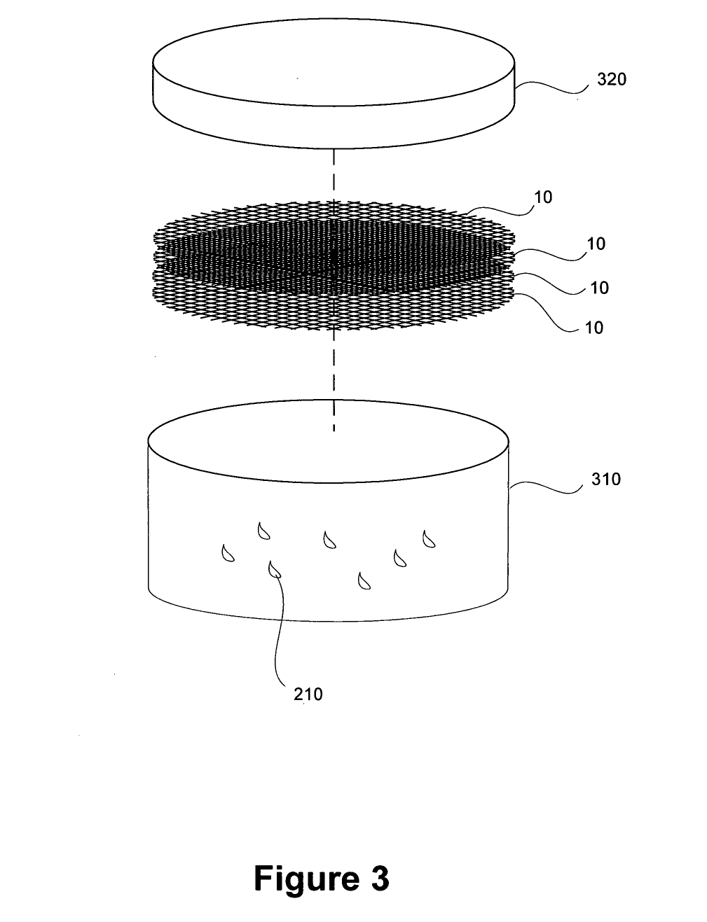

FIG. 3 is a sectional view of another assembly in which disposable, absorbent pads may be packaged according to one embodiment of the present invention. According to one embodiment of the present invention, as shown in FIG. 3, the assembly may be a resealable container 310 whereby multiple absorbent pads 10 may be stored with healing solution 210. It is understood that the resealable container 310 may be of varying shapes and sizes. The resealable container 310 may be unsealed and sealed again by screwing on and off a threaded lid 320. According to one embodiment of the present invention the threaded lid 320 may be constructed such that the resealable container 310 cannot be opened by a child. Moreover, the resealable container and lid may be constructed sealed and unsealed by methods other than screwing on and off, such as popping or pressing on and off, or zip closure.

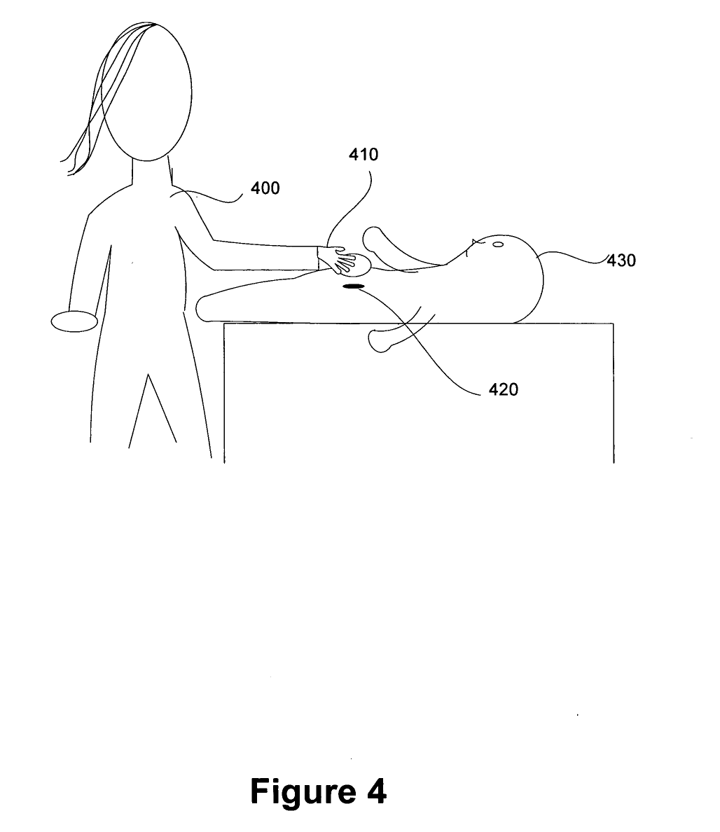

FIG. 4 is a drawing of an application technique for one embodiment of the present invention. FIG. 4 shows the user 400 holding a disposable absorbent pad 410. The user 400 is applying the disposable pad 410 to the area surrounding the umbilical stump 420 of a baby 430 in order to facilitate healing.

The present invention is not to be limited in scope by the specific exemplary embodiments described herein. Indeed, various modifications of the present invention, in addition to those described herein, will be apparent to those of ordinary skill in the art from the foregoing description and accompanying drawings. Thus, such modifications are intended to fall within the scope of the following appended claims. Accordingly, the claims set forth below should be construed in view of the full breath and spirit of the present invention as disclosed herein.

What is claimed is:

1. An assembly for the treatment of an umbilical stump comprising:
a disposable absorbent pad;
a healing solution for treatment of an area surrounding an umbilical stump of a baby;
and a packaging assembly.

2. The assembly of claim 1 wherein the disposable absorbent pad is a woven material.

3. The assembly of claim 1 wherein the disposable absorbent pad is a non-woven material.

4. The assembly of claim 1 wherein the healing solution is a solution comprised of witch hazel.

5. The assembly of claim 1 wherein the healing solution is a solution consisting of:
witch hazel; and
at least one of a fruit extract and an herb extract.

6. The assembly of claim 1 wherein the healing solution is a solution consisting of:
witch hazel;
black willow bark extract; and
at least one of a fruit extract and an herb extract.

7. The assembly of claim 1 wherein the packaging assembly comprises:
a jar; and
a re-sealable lid to the jar.

8. The assembly of claim 7 wherein the jar and the re-sealable lid cannot be opened by a child.

9. The assembly of claim 1 wherein the packaging assembly comprises:
a sealed single-use packet capable of retaining the healing solution contained until the packet is opened in order to apply the absorbent to the umbilical stump.

10. The assembly of claim 9 wherein the sealed single-use packet can be torn open by an adult.

11. The assembly of claim 2 wherein the healing solution is a solution comprised of witch hazel.

12. The assembly of claim 2 wherein the healing solution is a solution consisting of:
witch hazel; and
at least one of a fruit extract and an herb extract.

13. The assembly of claim 2 wherein the healing solution consisting of:
witch hazel;
black willow bark extract; and
at least one of a fruit extract and an herb extract.

14. The assembly of claim 3 wherein the healing solution is a solution comprised of witch hazel.

15. The assembly of claim 3 wherein the healing solution is a solution consisting of:
witch hazel; and
at least one of a fruit extract and an herb extract.

16. The assembly of claim 3 wherein the healing solution consisting of:
witch hazel;
black willow bark extract; and
at least one of a fruit extract and an herb extract.

17. A method of treating an umbilical stump comprising:
applying a disposable, absorbent pad previously moistened with a healing solution to the area surrounding an umbilical stump of a baby, without applying the healing solution to the disposable, absorbent pad at the time of application to the area surrounding the umbilical stump.

18. A solution to aid in the healing of the umbilical stump consisting of:
a mild astringent for application on an umbilical stump;
at least one of a fruit extract and an herb extract; and
a preservative.

19. A solution to aid in the healing of the umbilical stump comprised of:
a mild astringent for application on an umbilical stump;
a healing accelerator; and
a preservative.

20. The solution of claim 20 wherein the healing accelerator consists of at least one of lavender, chamomile, and aloe.
